(19) United States
(12) Patent Application Publication
(10) Pub. No.: US 2021/0401379 A1
(43) Pub. Date: Dec. 30, 2021

(54) EARLY WARNING SCORE DESIGN AND OTHER SCORES

(71) Applicant: KONINKLIJKE PHILIPS N.V., EINDHOVEN (NL)

(72) Inventors: SABINE FUNKE-SCHAEFF, AIDLINGEN (DE); CORRADO GRIECI, APEX, NC (US)

(21) Appl. No.: 17/282,795

(22) PCT Filed: Oct. 8, 2019
(86) PCT No.: PCT/EP2019/077135

§ 371 Date: Apr. 5, 2021

Related U.S. Application Data
(60) Provisional application No. 62/746,012, filed on Oct. 16, 2018.

(51) Int. Cl. A61B 5/00; G16H 40/63; G16H 50/30; A61B 5/021; A61B 5/024; A61B 5/08

(52) U.S. Cl. CPC A61B 5/743; G16H 40/63; G16H 50/30; A61B 5/021; A61M 2230/40; A61B 5/08; A61B 5/7475; A61M 2230/30; A61M 2230/50; A61B 5/024

(57) ABSTRACT

The following relates to medical device technology. In one aspect, a score is calculated based on a patient's vital sign information. The score is displayed in a center circle. Spokes corresponding to individual vital signs may extend from the center circle. The spokes may be color coded based on a subscore (e.g. severity level) of the vital sign that they correspond to. A thickness of each spoke may be based on subscore of the corresponding vital sign.

### BACKGROUND

The following relates generally to medical equipment technology, and more specifically to medical display devices.

Current patent monitoring systems provide an Early Warning Score (EWS) display. A standard EWS such as the Modified EWS (MEWS) is an illness assessment score based on vital signs typically including pulse, respiration rate, blood pressure, body temperature, and a level of consciousness metric (e.g., the alert/voice/pain/unresponsive or AVPU standard which assigns 0 to +3 points on this scale). In MEWS, a normal vital sign reading is assigned a score of 0, and increasingly abnormal readings (e.g. increasingly above or below the normal pulse range) are assigned progressively higher integer values, and the final MEWS score is the sum of these values. Hence, a higher MEWS score indicates higher patient criticality. However, the detailed EWS formulation varies with different hospitals (for example, SpO₂ and/or urinary output in last two hours may be an additional vital sign). In this regard, it should be noted that there are other variants of EWS as well, such as National Early Warning Score (NEWS) (e.g., EWS is one protocol that may be used, and NEWS is another protocol that may be used). The EWS scores for all patients in a hospital ward may be displayed on the nurses' station dashboard as a grid, or may be displayed in a scrollable list on an associated mobile device app. The EWS scores are also used as an alarm triggering system.

However, an elevated EWS score by itself conveys limited information, as it does not indicate which abnormal vital sign(s) are causing the EWS elevation. Typically, medical personnel must review the patient's current/recent vital sign readings to detect the source of the elevated EWS score. This takes valuable time in what may be an acute care situation.

The following provides a new and improved systems and methods which overcome the above-referenced problems and others.

### SUMMARY

In one disclosed aspect, a medical display device includes: a display screen; at least one electronic processor; and at least one memory including computer program code. The at least one memory and the computer program code may be configured to, with the at least one processor, cause the medical display device to: display, on the display screen, in a center circle, a score calculated based on a patient's: blood pressure, SpO₂ level, heart rate, respiratory rate, and temperature; and display, on the display screen, at least one color coded spoke extending from the center circle. The at least one color coded spoke: corresponding to one of the patient's blood pressure, SpO₂ level, heart rate, respiratory rate, and temperature; and color coded based on a subscore of the corresponding blood pressure, SpO₂ level, heart rate, respiratory rate, and temperature.

In some embodiments, the at least one processor is configured to execute the computer-readable instructions to cause the medical display device to calculate the score further based on the patient's level of consciousness. In some embodiments, the at least one processor is configured to execute the computer-readable instructions to cause the medical display device to receive the patient's level of consciousness from an input device configured to receive the level of consciousness from a caregiver. In some approaches, the at least one color coded spoke comprises at least five color coded spokes; each of the five color coded spokes correspond to one of the patient's blood pressure, SpO₂ level, heart rate, respiratory rate, and temperature; and the at least one processor is configured to execute the computer-readable instructions to cause the medical display device to: color code the five color coded spokes based on a subscore of the corresponding blood pressure, SpO₂ level, heart rate, respiratory rate, and temperature; and display the five color coded spokes. In some embodiments, the at least one processor is configured to execute the computer-readable instructions to cause the medical display device to: increase a thickness of the at least one color coded spoke when the subscore increases; and decrease the thickness of the at least one color coded spoke when the subscore decreases.

In some embodiments, the at least one color coded spoke comprises a heart rate spoke and a respiratory rate spoke; and the at least one processor is configured to execute the computer-readable instructions to cause the medical display device to display: a heart rate symbol on the heart rate spoke; and a lung symbol on the respiratory rate spoke. In some embodiments, the at least one color coded spoke comprises a blood pressure spoke, a temperature spoke and a SpO₂ spoke; and the at least one processor is configured to execute the computer-readable instructions to cause the medical display device to display: a meter symbol on the blood pressure spoke; a thermometer symbol on the temperature spoke; and a water droplet symbol on the SpO₂ spoke. In some embodiments, the score is a first score calculated using a first protocol; and the at least one processor is configured to execute the computer-readable instructions to cause the medical display device to: re-color code the at least one color coded spoke based on a second score calculated using a second protocol; and allow a user to: switch between displaying the first score and the second score, and display the first score and second score simultaneously. In some embodiments, the at least one processor is configured to execute the computer-readable instructions to cause the medical display device to allow a user to switch to an overview view, wherein the overview view: shows multiple patients with corresponding scores, previous scores and subscores. In some embodiments, the at least one processor is configured to execute the computer-readable instructions to cause the medical display device to receive patient's vital signs including at least: blood pressure from a blood pressure measuring device, SpO₂ level from a pulse oximeter, heart rate from a heart rate monitor, respiratory rate from a respiratory monitor, and temperature from a thermometer; and to determine a subscore for each received vital sign based on an extent of deviation of the received vital sign from a normal range for the vital sign and to calculate the score by summing the subscores. In some embodiments, the at least one processor is configured to execute the computer-readable instructions to cause the medical display device to: determine that at least one of the patient's blood pressure, SpO₂ level, heart rate, respiratory rate, and temperature is in a normal range; and not display a color coded spoke corresponding to the determined at least one of the patient's blood pressure, SpO₂ level, heart rate, respiratory rate, and temperature that is in the normal range.

In another disclosed aspect, a medical display device includes: at least one processor; and at least one memory including computer program code. The at least one memory and the computer program code may be configured to, with the at least one processor, cause the medical display device to: display, in a center circle, a score calculated based on a plurality of vital signs of a patient; and display a color coded spoke extending from the center circle, the color coded spoke corresponding to a first vital sign of the plurality of vital signs; wherein the color coded spoke is color coded based on a subscore of the first vital sign; and wherein a thickness of the color coded spoke depends on the subscore of the first vital sign.

In a medical display device as described in the preceding paragraph, in some embodiments, the first vital sign is blood pressure, and the color coded spoke is a first color coded spoke corresponding to the blood pressure; the plurality of vital signs of the patient further includes: a second vital sign of SpO₂, a third vital sign of heart rate, a fourth vital sign of respiratory rate, and a fifth vital sign of temperature; and the at least one memory and the computer program code configured to, with the at least one processor, cause the medical display device to display: a second color coded spoke, wherein the second color coded spoke is color coded based on a subscore of the SpO₂, and a thickness of the second color coded spoke depends on the subscore of the SpO₂; a third color coded spoke, wherein the third color coded spoke is color coded based on a subscore of the heart rate, and a thickness of the third color coded spoke depends on the subscore of the heart rate; a fourth color coded spoke, wherein the fourth color coded spoke is color coded based on a subscore of the respiratory rate, and a thickness of the fourth color coded spoke depends on the subscore of the respiratory rate; and a fifth color coded spoke, wherein the fifth color coded spoke is color coded based on a subscore of the temperature, and a thickness of the fifth color coded spoke depends on the subscore of the temperature.

In another disclosed aspect, a method, performed by at least one electronic processor, includes: displaying, in a center circle, a score calculated based on a plurality of vital signs of a patient; and displaying a color coded spoke extending from the center circle, the color coded spoke corresponding to a first vital sign of the plurality of vital signs. The color coded spoke may be color coded based on a subscore of the first vital sign; and a thickness of the color coded spoke may depend on the subscore of the first vital sign.

One advantage resides in a medical display device that displays more important medical information in a smaller display area than in previous systems.

Another advantage resides in displaying information in a way that a caregiver is more easily able to understand in a shorter amount of time. Hence, the caregiver is enabled to make a critical care decision in a shorter amount of time.

Another advantage resides in displaying information in a way that is customized more specifically to how a particular caregiver may want to view particular information.

Other advantages will become apparent to one of ordinary skill in the art upon reading and understanding this disclosure. It is to be understood that a specific embodiment may attain, none, one, two, more, or all of these advantages.

The invention may take form in various components and arrangements of components, and in various steps and arrangements of steps. The drawings are only for purposes of illustrating the preferred embodiments and are not to be construed as limiting the invention.

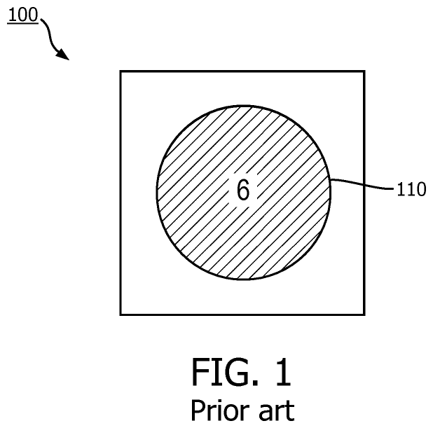

FIG. 1 shows an example of a prior system.

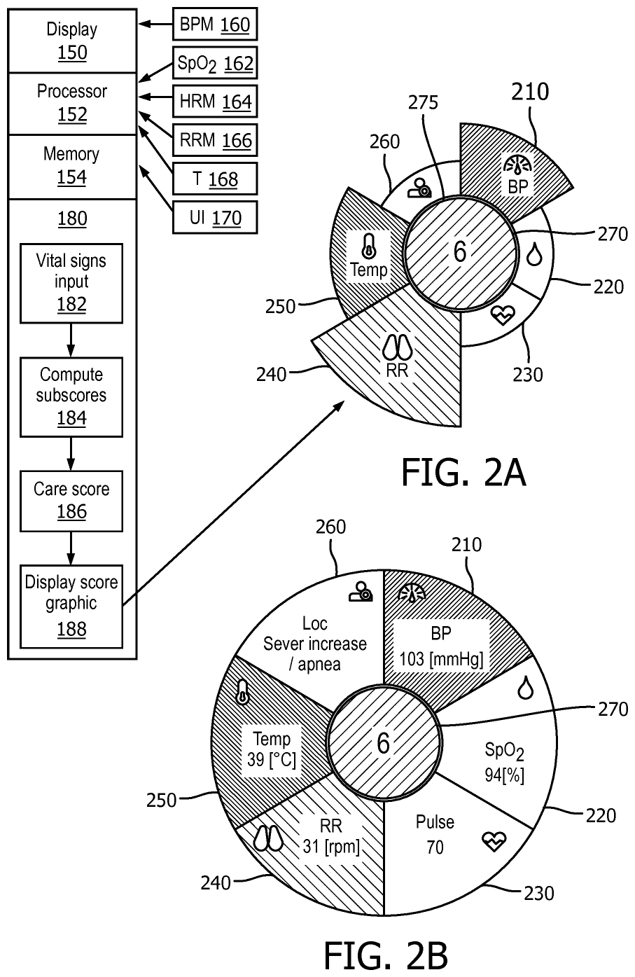

FIGS. 2A and 2B show embodiments of the systems and methods described herein.

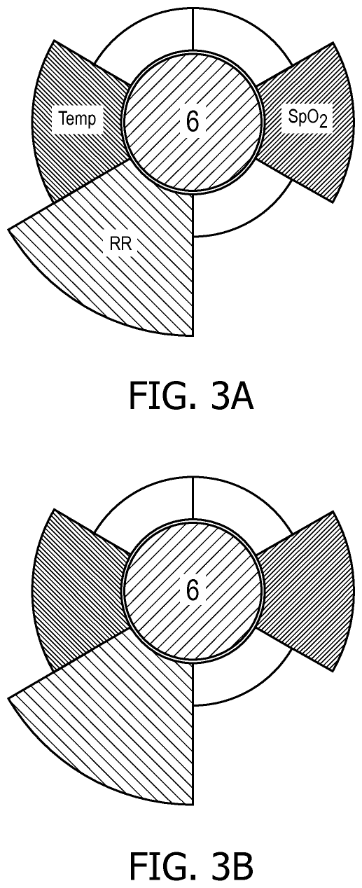

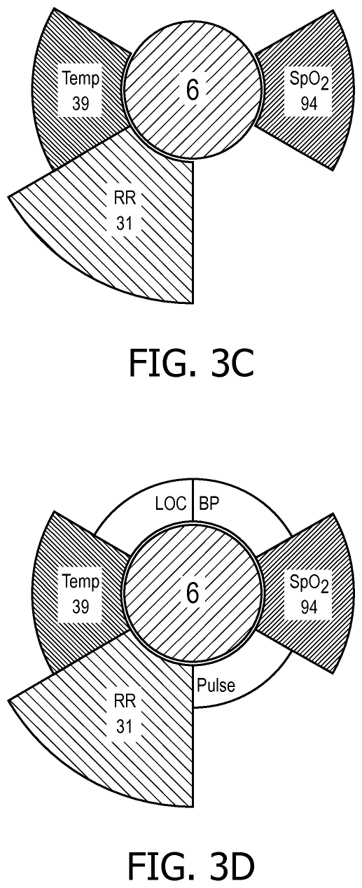

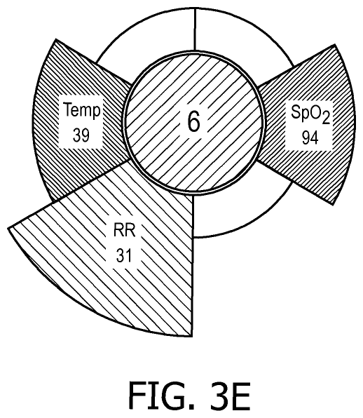

FIGS. 3A-3E show examples of aspects of the present invention.

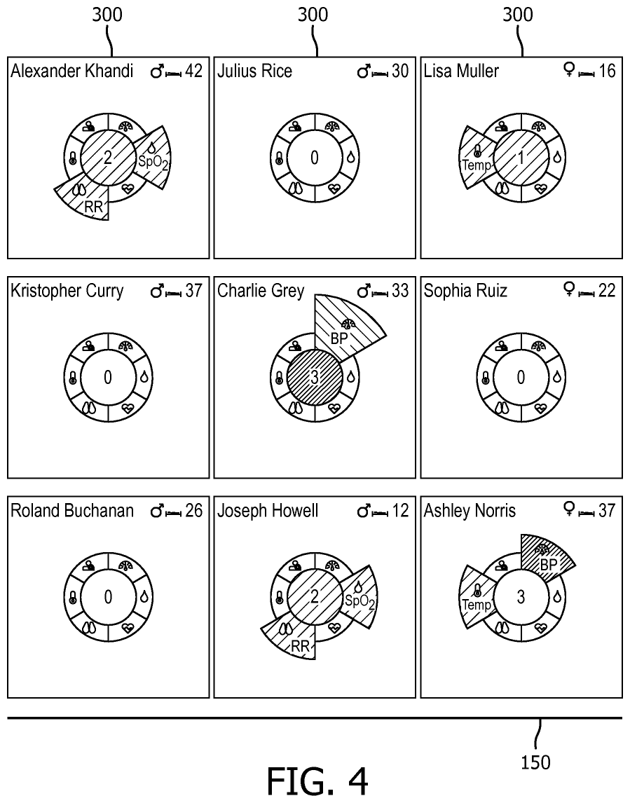

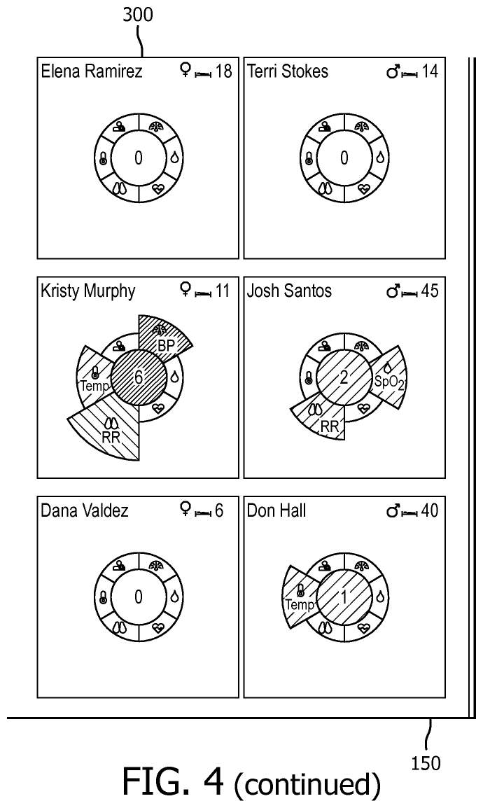

FIG. 4 illustrates an example of an embodiment displaying information for multiple patients.

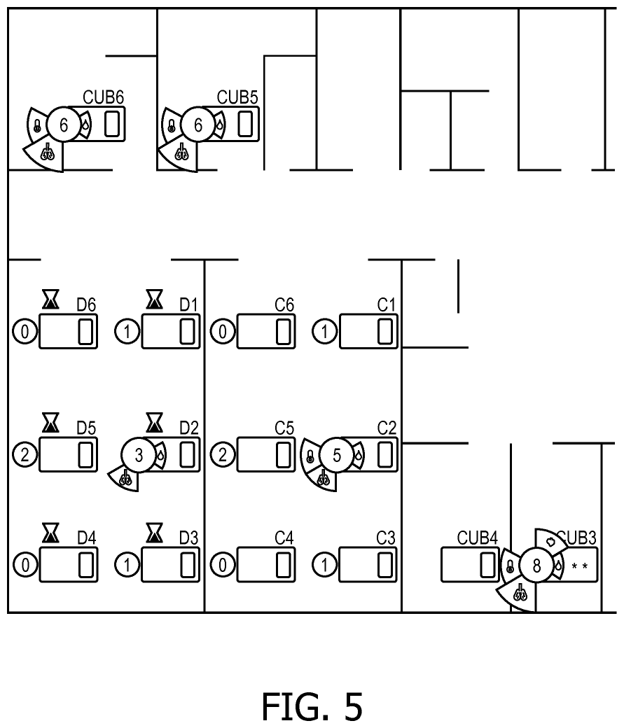

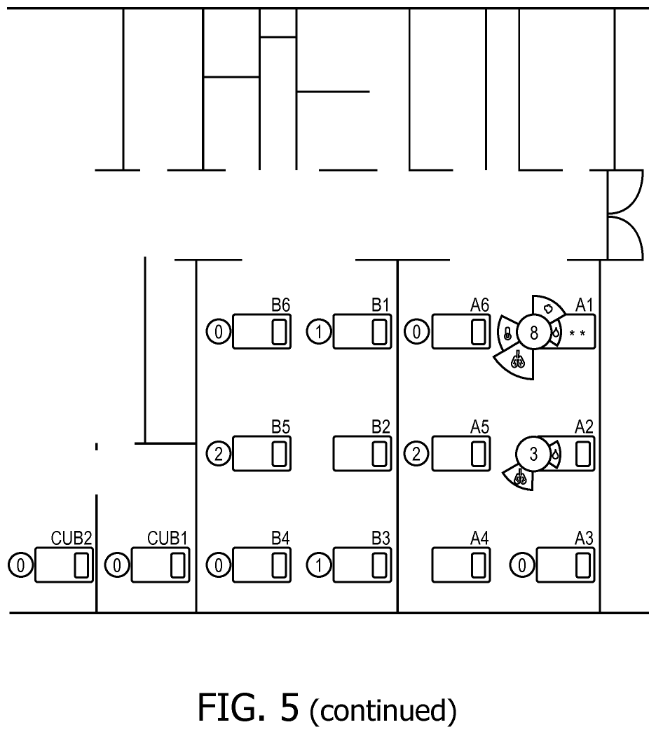

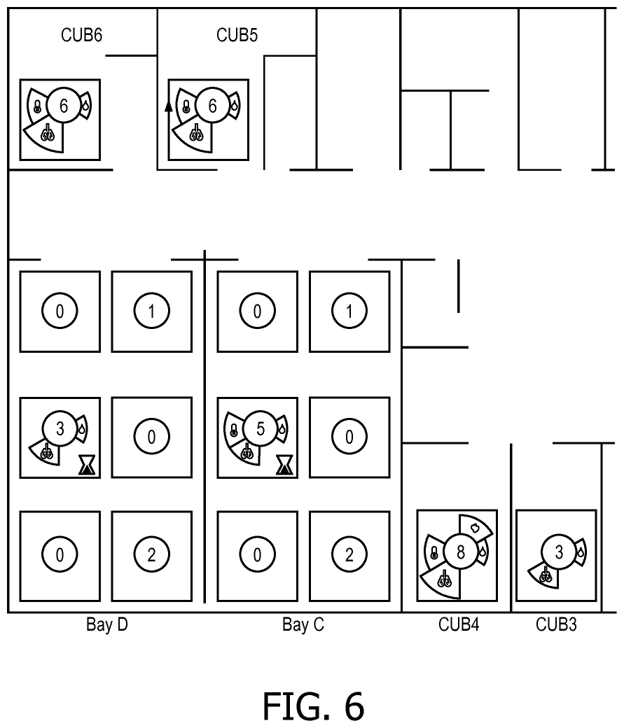

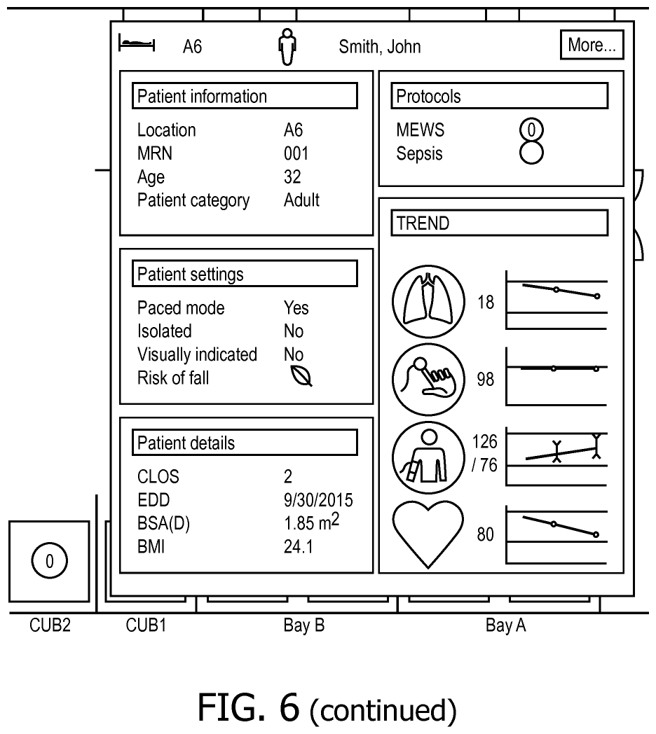

FIGS. 5 and 6 illustrate examples of embodiments including displaying patient information based on a floorplan.

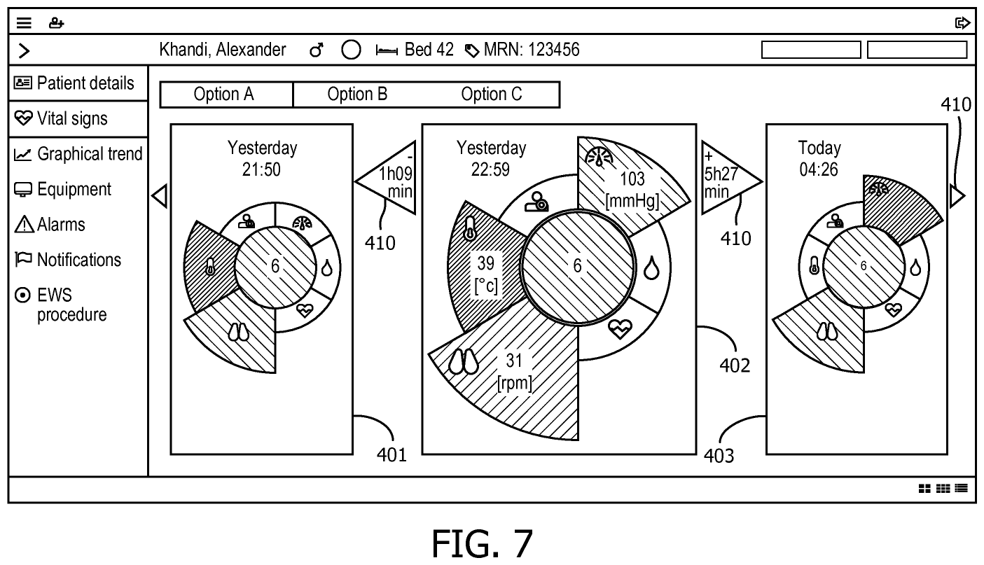

FIG. 7 illustrates an example of a patient workbook.

### DETAILED DESCRIPTION

The approaches disclosed herein show an EWS score and physiological data details of a patient. This EWS score display provides the caregiver with more detailed information which may assist to reduce mortality and mitigate severe adverse events in the hospital. The EWS score display as disclosed herein uses the cognitive translation of the brain to obtain the correct and relevant data in a single glance.

In previous systems, as shown in FIG. 1, the EWS display 100 is shown in a circle with the EWS number and the EWS color 110. In FIG. 1, the color 110 is depicted as shading, and it should be understood that throughout the figures various colors may be shown as shading patterns. In one example, the color 110 may be orange (represented in FIG. 1 as a diagonally crossed shading pattern). Typically, caregiver response rules specify whom to inform (e.g., physician or rapid response team member) depending on the number and color of the EWS display 100. With some hospital workflow and process rules, the caregiver is also advised by the EWS number and color when the next EWS measurement and calculation should be done to observe the patient behavior and deterioration. Using the prior EWS display 100 of FIG. 1, the caregiver is informed of the criticality of the patient but does not know which physiological data, laboratory data or clinician assessment are out of the normal range. The prior systems do not show the sub scores for the various vital signs that contribute to the overall EWS score, and do not show the body system and organ connected with such out of range measurement/assessment.

In contrast to the prior systems, with the systems and methods described herein, the caregiver obtains the information about the vital sign, lab data or caregiver assessment inputs. Also shown is the subscores of these values and also the risk color of these values. The body system is also shown or indicated as well, which allows a clinician to determine, for example, that the deterioration is coming from the lung, the heart etc.

To further illustrate, the systems and methods described herein include various improvements to facilitate rapid and fuller comprehension of the patient status. In one aspect, an improved EWS score representation is provided, which places the EWS score at the central hub of a wheel graphic, color coded to indicate severity (e.g., white may indicate a normal vital sign; while yellow, orange, and red indicate progressively increasing severity of the vital sign). Six spokes or arcs of the wheel then represent the various vital signs: heart rate (HR); respiration rate (RR); blood pressure (BP); peripheral capillary oxygen saturation (SpO₂); temperature (Temp); and level of consciousness (LOC), and are color-coded to indicate the severity of each respective vital sign. In some embodiments, alert, voice, pain, unresponsive (AVPU) is used instead of LOC. In addition, it should be noted that LOC may be a measurement of the patient's responsiveness to stimuli from the environment, and may be measured by a caregiver and input to an input device by the caregiver.

FIGS. 2A and 2B show embodiments of the systems and methods described herein. The system includes a display screen 150, an electronic processor 152, and at least one memory 154. The display screen 150 may be (by way of non-limiting illustrative example) an LCD display screen, an OLED display screen, a plasma display screen, a cathode ray tube (CRT), or so forth, any may be a standalone display screen or maybe incorporated into a patient monitor, nurses' station electronic white board, mobile device (e.g. cellphone or tablet computer), desktop or notebook computer, or so forth. The electronic processor 152 may be (by way of non-limiting illustrative example) a microprocessor or microcontroller and ancillary electronics such as power circuitry, biasing resistors, and/or so forth, and may in some embodiments comprise the central processing unit (CPU) of a patient monitor, nurses' station electronic white board, mobile device, desktop or notebook computer, or so forth. The at least one memory 154 may (by way of non-limiting illustrative example) comprise one or more of a random access memory (RAM) integrated circuit (IC), a read-only memory (ROM) IC, a flash memory IC, and/or so forth, and may in some embodiments comprise on-board or connected storage of a patient monitor, nurses' station electronic white board, mobile device, desktop or notebook computer, or so forth. It is also contemplated for the display screen 150, the electronic processor 152, and the memory 154 to be variously distributed. For example, the electronic processor 152 could be embodied by a network server computer located in an information technology (IT) department or the like, while the display screen 150 may be located in a doctor's office, at a nurses' station, or in some other clinical setting and operatively connected with the electronic processor 152 via a hospital network or the like. In another example, the electronic processor 152 may again be embodied as a server computer while the display screen 150 may be the display of a cellphone operatively connected with the server via a 4G or other cellular network. Still further, it will be appreciated that the may be various multiplicities of the components, e.g. there may be a number of display screens 150 distributed around the hospital (e.g., nurses station whiteboard display, bedside patient monitor display, physician's cellphone display, personal computer accessing the server as a website via an Internet connection, and/or so forth) all operatively connected with the electronic processor 152 in the form of the network server computer. These are merely non-limiting illustrative examples.

The electronic processor 152 is operatively connected (e.g. via a wired connector or a wireless link such as a Bluetooth™ link) to read one or more vital sensors, such as (by way of non-limiting illustrative example): a blood pressure (BP) monitor 160 (e.g. a blood pressure cuff, invasive arterial blood pressure probe, or so forth) from which a blood pressure of a patient being monitored is received; a pulse oximeter 162 from which a blood oxygenation (SpO₂) value of the patient being monitored is received; a heart rate monitor 164 (e.g. an electrocardiograph or the like) from which a heart rate of the patient being monitored is received; a respiratory monitor 166 (e.g. a respiratory monitoring belt) from which a respiratory rate of the patient being monitored is received; and a thermometer 168 (e.g. an oral thermometer, rectal thermometer, core body temperature thermometer, or so forth) from which a temperature of the patient being monitored is received. Additionally or alternatively, the system may include a user interface (UI) 170 (e.g. a keyboard, keypad, soft keyboard or keypad displayed on the display screen 150 which in such case is a touch-sensitive display screen 150, mouse, and/or so forth; optionally operating in conjunction with the display screen 150 under control of the electronic processor 152) via which a user (e.g. a nurse, doctor, or other medical caregiver) may enter one or more vital sign readings. For example, a nurse may enter a heart rate determined by the nurse via palpitation via the UI 170, and/or may enter a respiratory rate determined by visually observing chest rise/fall cycling, and/or may enter an SpO₂ reading obtained using a fingertip pulse oximeter that is not operatively connected with the electronic processor 152, and/or may enter a blood pressure reading (e.g. including both systolic and diastolic components) determined by the nurse using a manual blood pressure cuff and connected reader, and/or may enter a temperature reading obtained using a manual oral or rectal thermometer, and/or may enter a level of consciousness (LOC) determined by observation of the patient in accord with the alert/voice/pain/unresponsive or AVPU standard or some other LOC standard, and/or so forth. In other embodiments, such as in the case of the electronic processor 152 being the CPU of a nurses' station electronic whiteboard, the various vital sign values may be received via an electronic network (not shown) such as a hospital WiFi network or Ethernet. Similarly, if the electronic processor 152 is the CPU of a mobile device then the vital signs may be received over a 4G or other wireless cellular network or via WiFi. These are merely illustrative examples.

The at least one memory 154 stores computer program code which when executed by the electronic processor 152 causes an early warning system (EWS) method 180 to be performed. The EWS method 180 includes an input operation 182 in which vital sign values are received from operatively connected vital sign sensors 160, 162, 164, 166, 168 and/or from the UI 170 and/or from a connected wired or wireless electronic network (not shown). In an operation 184, the subscore for each vital sign is determined in accord with a chosen early warning system protocol. By way of non-limiting illustrative example, in a modified early warning system (MEWS) protocol, a subscore for each received vital sign is determined based on an extent of deviation of the received vital sign from a normal range for the vital sign. In this illustrative example, the systolic BP should be in the range 101-199 mmHg which has subscore=0; a low systolic reading of 81-100 mmHg is scored +1, a low systolic reading of 71-80 mmHg is scored +2, a low systolic reading of less than 70 mmHg is scored +3, and a high systolic reading of 200 mmHg or higher is scored +2. In this illustrative example, the heart rate should be in the range 51-100 beats per minute (bpm) which has subscore=0; a low pulse of 41-50 bpm or a high reading of 101-110 bpm scores +1; a low pulse of less than 40 bpm or a high pulse of 111-129 bpm scores +2; and a high pulse of 130 bpm or higher scores +3. In this illustrative example, the respiratory rate should be in the range 9-14 breaths per minute (bpm) which has subscore=0; a low rate of less than 9 bpm or a high rate of 15-20 bpm scores +1; a high rate of 21-29 bpm scores +2; and a high rate of 30 bpm or higher scores +3. For temperature, a normal reading in the range 35-38.4 degrees Celsius scores subscore=0, while any temperature above or below this range scores +2. Using the AVPU scale for LOC, a level of “alert” has subscore=0; a level of “voice reaction” has subscore=1; a level of “pain reaction” has subscore=2; and a level of “unresponsive” has subscore=3. Again, these subscore scales are merely illustrative examples, and other subscore assignment protocols may be used. Moreover, in some embodiments the system is programmed to implement two or more different protocols, and the user (e.g. nurse or doctor) can select which protocol to use via the UI 170. For example, a different protocol may be used for infants or other specialized types of patients, and/or for specific ailments such as In an operation 186, the score is calculated from the subscores. In a typical MEWS protocol, this is done by summing the subscores, although other aggregation formulas are contemplated depending upon the implemented early warning system protocol, e.g. applying different weighting values to the subscores.

Finally, in an operation 188, a MEWS or other early warning system score graphic is displayed. The illustrative case of FIG. 2A shows BP spoke 210, SpO₂ spoke 220, HR spoke 230, RR spoke 240, Temp spoke 250, level of consciousness (LOC) spoke 260, and EWS 270. In one aspect, the EWS 270 is displayed in the center circle 275. In one aspect, as shown in FIG. 2A, the shading pattern of Temp spoke 250 represents yellow; the shading pattern of BP spoke 210 and EWS 270 represents orange; and the shading pattern of RR spoke 240 represents red. In the examples of FIGS. 2A and 2B, the EWS score is 6 and the color is orange (represented by the shading of EWS 270), which means a warning. In these examples, at a glance, the caregiver understands that the main vital sign forcing the high EWS score is the RR with a value of 31 (Respiration) and with a red color. In one aspect, the caregiver knows the red color corresponds to a sub score +3. As illustrated in FIG. 2B, the second vital sign Temp 39 degree is the orange color, which differs from the normal value with the white color. In one embodiment, an SpO₂ value of less than 94 is below the normal SpO₂ value and is illustrated as yellow; in accordance with this embodiment, in the example of FIG. 2B, the SpO₂ is illustrated as white because it is 94.

In a further aspect also shown in FIG. 2A, the thickness and/or length of the spokes 210, 220, 230, 240, 250, 260 depends on the subscore number. Hence, the RR, with its high +3 subscore, extends further out from the central “hub” showing EWS score 270 compared with the other vital signs with lower subscores. Somewhat similarly, the color of each segment 210, 220, 230, 240, 250, 260 depends on the subscore. In one aspect, the subscore is a severity level of a corresponding blood pressure, SpO₂ level, heart rate, respiratory rate, temperature, or LOC.

In yet another aspect, the detailed representation and amount of information displayed in the spokes 210, 220, 230, 240, 250, 260 depends on the size of the EWS representation. For a dashboard grid in which each patient is represented by a small grid rectangle, the segments 210, 220, 230, 240, 250, 260 may only be color coded and sized (e.g. by thickness and/or length) to indicate severity (e.g., a larger thickness highlighting more severely out-of-range vital signs), and perhaps labeled with vital sign icons. Segments for vital signs in the normal range may be omitted to further save space and focus the clinician's attention on the out-of-range vital sign(s) (see, e.g., the example of FIG. 3C). On the other hand, a larger representation may annotate each spoke 210, 220, 230, 240, 250, 260 with the numerical value and/or textual label for the vital sign. When a patient deteriorates, the Score graphic shows at least one segment with no “normal” color and thickness.

To elaborate, the vital sign values correspond to segments (e.g. the spokes 210, 220, 230, 240, 250, 260 of FIG. 2A), and the number of segments are the number of values which are part of the Early Warning Score. The values (e.g. the RR of 31 shown in FIG. 2B) are vital signs, lab data, behavior, caregiver inputs, other scores and so forth. The score calculation follows the configured protocol setup. The color and subscores, and the limit and risk definition are customizable.

In some embodiments, the label of the vitals on the screen are placed at the column or row and are placed in the circle in a specified place. This place order can be configured in the Protocol Configuration of the system. Many scoring protocols may be defined and selectively implemented by a given system, for example as selected by a user via the UI 170, or as pre-configured by the vendor during installation of the EWS system at a given hospital or medical facility based on the country of installation or the early warning system protocol provided by the customer.

Another optional aspect is to provide a progressively more detailed drill-down of information. For example, starting with the dashboard grid or scrolling list of EWS scores for patients of a hospital ward, selection of one patient by clicking with a mouse or via a touch-screen causes a new screen showing that patient's information using a more detailed EWS graphical representation and listing values of only out-of-range vital sign(s). A further selection of a “Details” button, tab or other GUI control brings up a new screen showing all vital signs of the patient (now including the normal vital sign readings).

In yet a further optional aspect, the amount of detail in the EWS representation may be dynamically scaled with size. For example, in a dashboard setting, if the window presenting the grid of EWS scores is small then the color-coded representation may be used, then if the window is resized to enlarge it (e.g. by clicking it to be full-screen, or by grabbing a corner handle using a mouse pointer and pulling to enlarge the window) then progressively more detailed representations may be shown, e.g. adding text labels and numerical values, and perhaps using progressively larger font size for these. Aspects of this are shown in FIGS. 3A-3E. For example, FIG. 3A shows a view with text labels for Temp, SpO₂, and RR (as these have the largest subscores), and in which the spokes are scaled in size based on the subscore of the represented vital sign, whereas FIG. 3B shows the same features except that the text labels are omitted. Hence, the example of FIG. 3B would be better suited for a display in which the EWS graphic is smaller (e.g., a cell representing a patient in an electronic whiteboard). FIG. 3C shows a variant embodiment in which only those vital signs whose subscore exceeds some threshold (e.g. +2 or higher) are shown in the example, only the temperature, RR, and SpO₂ spokes are shown, while the BP, HR (i.e. pulse), and LOC spokes are omitted as they have lower subscores. FIG. 3D shows a graphic which differs from that of FIG. 3C in that the BP, HR, and LOC with the lower subscores are indicated by residual spokes labeled only with identification of the respective vital signs. The graphic of FIG. 3E is the same as that of FIG. 3D except that the labels on the residual spokes are omitted.

In yet another optional aspect, the specific EWS graphical representation may depend on the clinical protocol selected. Hence, there may be different EWS graphical representations for standard MEWS, for the Systemic Inflammatory Response Syndrome (SIRS) sepsis protocol, for the Sepsis-related organ failure assessment score (SOFA) protocol, and/or so forth. Optionally, different color coding schemes may be used for the different protocols. GUI controls may be provided to enable the clinician to switch between the different types of EWS scoring systems/representations, and/or two or more different EWS graphical representations for different protocols may be shown simultaneously.

In yet another optional aspect, the body system may be illustrated. For example, lung, or heart symbols or labels may be used to emphasize the body system. In other examples, a meter symbol may be displayed on the BP spoke, a thermometer symbol may be displayed on the temperature spoke, and a water droplet symbol may be displayed on the SpO₂ spoke (see, e.g., FIGS. 2A and 2B).

With reference to FIGS. 4-7, some examples of patient overview displays are shown, such as might be displayed, for example, on an electronic whiteboard or floor monitor computer at a nurses' station at an intensive care unit (ICU) or other critical care ward or floor. The goal here is to represent the current status of each patient in a way that can be rapidly assessed by medical personnel.

FIG. 4 illustrates an example of an embodiment displaying information for multiple patients on the display screen 150. The display is a grid of patient cells 300, each representing a single patient. Because the individual cells 300 are small in area, the EWS graphics used are preferably designed to be easily read in this small size, e.g. by using symbols to label the vital signs of the spokes and by not including (or minimizing the amount of) textual details.

FIGS. 5 and 6 illustrate examples of embodiments including displaying patient information based on a floorplan. In this case, the cells 300 of FIG. 4 are replaced by diagrammatic representations of the patient rooms of the critical care floor or ward. In some embodiments, it is contemplated to be able to switch between the grid view of FIG. 4 and a floor plan view of FIG. 5 or FIG. 6 by user operation of the UI 170.

FIG. 7 illustrates another contemplated display embodiment, in which the temporal progression of a single patient is displayed as a patient workbook. In this example, each day's status is displayed by an EWS graphic 401, 402, 403 for that day, and the user can scroll horizontally through the days using scroll buttons 410 or the like. The EWS graphic that is centered by the horizontal scrolling, namely the graphic 402 representing the EWS score of the patient as of “Yesterday 22:59” is shown larger, but the two neighboring days (graphics 401, 403) are shown in smaller size. In this way, the clinician can readily examine the EWS score for one particular day (via large graphic 402 in the example) and also immediately visually grasp whether the various vital signs of the patient are improving or degrading by looking at the temporally preceding EWS graphic 401 and the temporally succeeding EWS graphic 403. (Of course, if the clinician were to scroll all the way to the right so that today's EWS graphic 403 is centered, then there would not be any temporally succeeding EWS graphic).

It will be further appreciated that the techniques disclosed herein may be embodied by a non-transitory storage medium storing instructions readable and executable by an electronic data processing device to perform the disclosed techniques. Such a non-transitory storage medium may comprise a hard drive or other magnetic storage medium, an optical disk or other optical storage medium, a cloud-based storage medium such as a RAID disk array, flash memory or other non-volatile electronic storage medium, or so forth.

Of course, modifications and alterations will occur to others upon reading and understanding the preceding description. It is intended that the invention be construed as including all such modifications and alterations insofar as they come within the scope of the appended claims or the equivalents thereof.

1. A medical display device, comprising:
a display screen;
at least one electronic processor;
and at least one memory storing non-transitory computer-readable instructions;
the at least one memory and the computer-readable instructions configured to, with the at least one processor, cause the medical display device to:
display, on the display screen, in a center circle, a score calculated based on a patient's:
blood pressure,
SpO2 level,
heart rate,
respiratory rate, and
temperature; and
display, on the display screen, at least one color coded spoke extending from the center circle, the at least one color coded spoke
corresponding to one of the patient's blood pressure, SpO2 level, heart rate, respiratory rate, and temperature; and
color coded based on a subscore of the corresponding blood pressure, SpO2 level, heart rate, respiratory rate, and temperature.

2. The medical display device of claim 1, wherein the at least one processor is configured to execute computer to cause the medical display device to calculate the score further based on the patient's level of consciousness; and optionally: to receive the patient's level of consciousness from an input device configured to receive the level of consciousness from a caregiver.

3. (canceled)

4. The medical display device of claim 1, wherein:
the at least one color coded spoke comprises at least five color coded spokes;
each of the five color coded spokes correspond to one of the patient's blood pressure, SpO2 level, heart rate, respiratory rate, and temperature; and
the at least one processor is configured to execute the computer-readable instructions to cause the medical display device to:
color code the five color coded spokes based on a subscore of the corresponding blood pressure, SpO2 level, heart rate, respiratory rate, and temperature; and
display the five color coded spokes.

5. The medical display device of claim 1, wherein the at least one processor is configured to execute the computer-readable instructions to cause the medical display device to:
increase a thickness of the at least one color coded spoke when the subscore increases; and
decrease the thickness of the at least one color coded spoke when the subscore decreases.

6. The medical display device 1, wherein:
the at least one color coded spoke comprises a heart rate spoke and a respiratory rate spoke; and
the at least one processor is configured to execute the computer-readable instructions to cause the medical display device to display:
a heart rate symbol on the heart rate spoke; and
a lung symbol on the respiratory rate spoke.

7. The medical display of device 1, wherein:
the at least one color coded spoke comprises a blood pressure spoke, a temperature spoke and a SpO2 spoke; and
the at least one processor is configured to execute the computer-readable instructions to cause the medical display device to display:
a meter symbol on the blood pressure spoke;
a thermometer symbol on the temperature spoke; and
a water droplet symbol on the SpO2 spoke.

8. The medical display device of claim 1, wherein:
the score is a first score calculated using a first protocol; and
the at least one processor is configured to execute the computer-readable instructions to cause the medical display device to:
re-color code the at least one color coded spoke based on a second score calculated using a second protocol; and
allow a user to:
switch between displaying the first score and the second score, and
display the first score and second score simultaneously.

9. The medical display device claim 1, wherein the at least one processor is configured to execute the computer-readable instructions to cause the medical display device to allow a user to switch to an overview view, wherein the overview view:
shows multiple patients with corresponding scores, previous scores and subscores.

10. The medical display device of claim 1, wherein the at least one processor is configured to execute the computer-readable instructions to cause the medical display device to receive patient's vital signs including at least:
blood pressure from a blood pressure measuring device,
SpO2 level from a pulse oximeter,
heart rate from a heart rate monitor,
respiratory rate from a respiratory monitor, and
temperature from a thermometer;
and to determine a subscore for each received vital sign based on an extent of deviation of the received vital sign from a normal range for the vital sign and to calculate the score by summing the subscores.

11. The medical display device of claim 10, wherein the at least one processor is configured to execute the computer-readable instructions to cause the medical display device to:
determine that at least one of the patient's blood pressure, SpO2 level, heart rate, respiratory rate, and temperature is in a normal range; and
not display a color coded spoke corresponding to the determined at least one of the patient's blood pressure, SpO2 level, heart rate, respiratory rate, and temperature that is in the normal range.

12. A system including:
the medical display device of claim 1; and one or more of:
a blood pressure measuring device configured to send a blood pressure to the medical display device;
a pulse oximeter configured to send a SpO₂ level to the medical display device;
a heart rate monitor configured to send a heart rate level to the medical display device;
respiratory rate from a respiratory monitor; and
a thermometer configured to send a temperature to the medical display device.

13.-16. (canceled)

17. A medical display device, comprising:
at least one processor; and
at least one memory storing non-transitory computer-readable instructions;
the at least one memory and the computer-readable instructions configured to, with the at least one processor, cause the medical display device to:
display, in a center circle, a score calculated based on a plurality of vital signs of a patient; and
display a color coded spoke extending from the center circle, the color coded spoke corresponding to a first vital sign of the plurality of vital signs;
wherein the color coded spoke is color coded based on a subscore of the first vital sign; and
wherein a thickness and/or length of the color coded spoke depends on the subscore of the first vital sign.

18. The medical display device of claim 17, wherein:
the first vital sign is blood pressure, and the color coded spoke is a first color coded spoke corresponding to the blood pressure;
the plurality of vital signs of the patient further includes:
a second vital sign of SpO2,
a third vital sign of heart rate,
a fourth vital sign of respiratory rate, and
a fifth vital sign of temperature; and
the at least one memory and the computer-readable instructions configured to, with the at least one processor, cause the medical display device to display:
a second color coded spoke, wherein the second color coded spoke is color coded based on a subscore of the SpO2, and a thickness of the second color coded spoke depends on the subscore of the SpO2;
a third color coded spoke, wherein the third color coded spoke is color coded based on a subscore of the heart rate, and a thickness of the third color coded spoke depends on the subscore of the heart rate;
a fourth color coded spoke, wherein the fourth color coded spoke is color coded based on a subscore of the respiratory rate, and a thickness of the fourth color coded spoke, depends on the subscore of the respiratory rate; and
a fifth color coded spoke, wherein the fifth color coded spoke is color coded based on a subscore of the temperature, and a thickness of the fifth color coded spoke depends on the subscore of the temperature.

19. The medical display device of claim 17, wherein the at least one processor is configured to execute the computer-readable instructions to cause the medical display device to:
determine that at least one of a patient's blood pressure, SpO2 level, heart rate, respiratory rate, and temperature is in a normal range; and
not display a color coded spoke corresponding to the determined at least one of the patient's blood pressure, SpO2 level, heart rate, respiratory rate, and temperature that is in the normal range.

20. A method, performed by at least one electronic processor, comprising:
displaying, in a center circle, a score calculated based on a plurality of vital signs of a patient; and
displaying a color coded spoke extending from the center circle, the color coded spoke corresponding to a first vital sign of the plurality of vital signs;
wherein the color coded spoke is color coded based on a subscore of the first vital sign; and
wherein a thickness of the color coded spoke depends on the subscore of the first vital sign.
